(19) United States
(12) Patent Application Publication
(10) Pub. No.: US 2015/0174160 A1
(43) Pub. Date: Jun. 25, 2015

(54) METHODS FOR TREATING, DIAGNOSING AND/OR MONITORING PROGRESSION OF OXO ASSOCIATED STATES

(71) Applicant: Jay PRAVDA, (US)

(72) Inventor: Jay Pravda, Brookline, MA (US)

(21) Appl. No.: 14/412,099

(22) PCT Filed: Jul. 2, 2013
(86) PCT No.: PCT/US13/49087

§ 371 Date: Dec. 30, 2014

Related U.S. Application Data
(60) Provisional application No. 61/748,698, filed on Jan. 3, 2013.
(60) Provisional application No. 61/691,787, filed on Aug. 21, 2012.
(60) Provisional application No. 61/667,719, filed on Jul. 3, 2012.

(51) Int. Cl. A61K 33/04; A61K 31/198; G01N 33/50; A61K 31/20

(52) U.S. Cl. CPC A61K 33/04; A61K 31/20; A61K 31/198; G01N 33/50

(57) ABSTRACT

Disclosed are methods for diagnosing, treating and/or monitoring clinical progression associated states, e.g., hypermetabolic associated states or sepsis associated states.

### RELATED APPLICATIONS

This application claims priority to U.S. Provisional Application No. 61/667,719, filed on Jul. 3, 2012, U.S. Provisional Application No. 61/691,787, filed on Aug. 21, 2012, and U.S. Provisional Application No. 61/748,698, filed on Jan. 3, 2013, the entire contents of each of which are hereby incorporated herein by reference.

### BACKGROUND

Oxo associated states are characterized by the elevated levels of reactive intermediates, such as hydrogen peroxide (H₂O₂), and/or by diminished levels of glutathione in the body of an afflicted subject. Oxo associated states may include hypermetabolic associated states, or conditions that can develop as a result of hyperactivation of the immune system, e.g., following bacterial or viral infection.

Septic shock is one example of a hypermetabolic associated state that is the most common cause of mortality in the intensive care unit, with a fatality rate that can rise to 80% for those developing multiple organ failure. Overall, a third of all patients developing septic shock die despite receiving antibiotics and supportive care. Even patients who survive sepsis associated conditions for the first month, have a 2.7 times higher mortality rate the first year and a 2.3 times higher mortality rate the next three years compared to persons of similar age, sex and co-morbidity (Storgaard et al. (2012) Scand. J. of Trauma, Resusc. and Emergency Med. 20(Suppl 2):P28).

Elevated levels of oxidizing agents and/or diminished levels of glutathione are thought to contribute to the pathogenesis of oxo associated states. In the case of septic shock, this is thought to occur through the progression of an exaggerated systemic hypermetabolic response. Multiple therapeutic efforts aimed at treating oxo associated states, e.g., septic shock, have been uniformly unsuccessful (Goldengerg et al. (2011) Sci. Transl. Med., 3(88):88ps25). Therefore, new methods for treating oxo associated states are needed.

### SUMMARY OF THE INVENTION

A recent and surprising finding has been the discovery that certain pathological conditions are associated with elevated levels of oxidizing agents, e.g., hydrogen peroxide (H₂O₂), and/or with diminished levels of glutathione in the body of an afflicted individual. Some of these pathological conditions comprise hypermetabolic associated states that are characterized by hyperactivation of an immune system.

Accordingly, the present invention provides methods for diagnosing, treating or monitoring clinical progression of an oxo associated state in a subject. In one embodiment, a method for treating an oxo associated state in a subject comprises administering to the subject an effective amount of an oxoprotective agent, such that said oxo associated state in said subject is treated. In some embodiments, the oxo associated state is also a hypermetabolic associated state. In certain embodiments, the oxoprotective agent is a reactive intermediate scavenging agent or a glutathione level restoring agent.

In some embodiments, the oxoprotective agent is an active sulfur compound. In certain embodiments, the active sulfur compound is not sodium 2-mercaptoethene sulfonate or disodium 2,2′-dithiobis ethane sulfonate. In some embodiments, the active sulfur compound is selected from a group consisting of a sulfide compound, a sulfite compound, a thiosulfate compound, a thionite compound, a thionate compound, an organic sulfur compound, or precursors, hydrates and mixtures thereof. In some embodiments, the active sulfur compound is a thiosulfate compound. In further embodiments, the thiosulfate compound is sodium thiosulfate, ammonium thiosulfate, calcium thiosulfate, potassium thiosulfate, silver thiosulfate, choline thiosulfate, gold sodium thiosulfate, magnesium thiosulfate hexahydrate, and thiosulfate hyposulfite. In a specific embodiment, the active sulfur compound is sodium thiosulfate. In some embodiments, the oxoprotective agent is administered in combination with alpha-lipoic acid. In a specific embodiment, the alpha-lipoic acid is R-dihydro lipoic acid.

In some embodiments, the oxoprotective agent is administered parenterally. In further embodiments, the oxoprotective agent, such as sodium thiosulfate, is administered intravenously. In some embodiments, the R-dihydro-lipoic acid is administered orally. In alternative embodiments, the oxoprotective agent, such as sodium thiosulfate, and the R-dihydro-lipoic acid are administered parenterally, e.g., intravenously, as a part of the same pharmaceutical composition.

In some embodiments, methods for diagnosing, treating and/or monitoring clinical progression of an oxo associated state in a subject are provided. In some embodiments, the oxo associated state is a hypermetabolic associated state, e.g., a sepsis associated state. In some embodiments, a method for treating a sepsis associated state in a subject comprises administering to said subject an effective amount of a first oxoprotective agent, such that the sepsis associated state is treated. In some embodiment, the first oxoprotective agent is also administered in combination with a second oxoprotective agent. In certain embodiments, the first oxoprotective agent and/or the second oxoprotective agent are each active sulfur compounds. In some embodiments, said active sulfur compounds are not sodium 2-mercaptoethene sulfonate or disodium 2,2′-dithiobis ethane sulfonate.

In some embodiments, the first oxoprotective agent and the second oxoprotective agent are each active sulfur compounds independently selected from the group consisting of a sulfide compound, a sulfite compound, a thiosulfate compound, a thionite compound, a thionate compound, an organic sulfur compound, or precursors, hydrates and mixtures thereof. In further embodiments, the first oxoprotective agent is a thiosulfate compound and the second oxoprotective agent is an organic sulfur compound. In a specific embodiment, the first oxoprotective agent is sodium thiosulfate and the second oxoprotective agent is N-acetylcysteine.

In some embodiments, the first oxoprotective agent and the second oxoprotective agent are both administered parenterally. In further embodiments, the first oxoprotective agent and the second oxoprotective agent are both administered intravenously.

In some embodiments, the first oxoprotective agent and the second oxoprotective agent are administered as parts of separate pharmaceutical compositions. In alternative embodiments, the first oxoprotective agent and the second oxoprotective agent are administered as parts of the same pharmaceutical composition. In some embodiments, the first and the second oxoprotective agents are administered in combination with R-dihydro-lipoic acid. In one embodiment, the alpha-lipoic acid is R-dihydro lipoic acid. In one embodiment, the alpha-lipoic acid is administered orally.

In some embodiments, the oxo associated state is a hypermetabolic associated state. In one embodiment, the hypermetabolic associated state is a sepsis associated state. In further embodiments, the sepsis associated state is Systemic Inflammatory Response Syndrome (SIRS), sepsis, severe sepsis or septic shock.

In some embodiments, the hypermetabolic associated state is associated with a viral infection. In a specific embodiment, the viral infection is Dengue fever, Dengue hemorrhagic fever or Dengue Shock Syndrome.

In some embodiments, the method for treating an oxo associated state in a subject comprises administering to said subject an effective amount of sodium thiosulfate, such that said oxo associated state in said subject is treated. In further embodiments, sodium thiosulfate is administered in combination with R-dihydro lipoic acid.

In another embodiment, the method for treating an oxo associated state in a subject comprises administering to said subject an effective amount of sodium thiosulfate in combination with N-acetylcysteine and R-dihydro lipoic acid, such that the oxo associated state in said subject is treated. In some embodiments, the present invention also provides a method for diagnosing an oxo associated state, the method comprising measuring the level of glutathione and/or the level of hydrogen peroxide in one or more bodily fluids of a subject suspected of having an oxo associated state. In some embodiments, the level of glutathione measured in a subject afflicted with an oxo associated state is lower than the level of glutathione in a healthy subject. In some embodiments, the level of hydrogen peroxide measured in a subject afflicted with an oxo associated state is higher than the level of hydrogen peroxide in a healthy subject.

In some embodiments, the present invention also provides a method for monitoring treatment of an oxo associated state, the method comprising measuring the level of glutathione and/or the level of hydrogen peroxide in a subject being treated for an oxo associated state. In some embodiments, the measurement of the level of glutathione and/or the level of hydrogen peroxide is repeated at least once during administration of the treatment.

In some embodiments, the present invention also provides a method for monitoring treatment of an oxo associated state in a subject, the method comprising measuring the level of one or more oxoprotective agents in the blood of said subject. In some embodiments, the oxoprotective agent is sodium thiosulfate, N-acetylcysteine or R-dihydro lipoic acid.

### BRIEF DESCRIPTION OF THE DRAWINGS

FIG. 1 is a schematic illustrating the proposed mechanism of pathogenesis of an oxo associated state through hypermetabolism.

FIG. 2 is a graph showing the amount of peroxides in serum of patients diagnosed with septic shock, as measured by the PEROXsay™ assay.

### DETAILED DESCRIPTION

### Agents

The present invention provides methods for diagnosing, treating and/or monitoring the clinical course of an oxo associated state in a subject. In some embodiments, the method for treating an oxo associated state in a subject comprises administering to said subject an effective amount of an oxoprotective agent, such that the oxo associated state in said subject is treated. In some embodiments, the oxoprotective agent is not sodium 2-mercaptoethene sulfonate (mesna) or disodium 2,2′-dithiobis ethane sulfonate (dimesna). In some embodiments, the oxo associated state is a hypermetabolic associated state.

As used herein, the term “oxoprotective agent” refers to an agent that protects other molecules from oxidation. Oxidation involves transfer of one or more electrons or hydrogen from a molecule to an oxidant. In some embodiments, the oxoprotective agent reacts with one or more oxidants, thereby preventing the oxidants from reacting with, and oxidizing molecules present in the environment. In some embodiments, the oxidants are reactive oxygen species (ROS) and reactive nitrogen species (RNS). In a specific embodiment, the oxidant is hydrogen peroxide (H₂O₂).

In some embodiments, the oxoprotective agent is a reactive intermediate scavenging agent. The reactive intermediate scavenging agent is an agent that scavenges reactive intermediates that may be present in a body of a subject afflicted with an oxo associated state, e.g., a hypermetabolic associated state. In some embodiments, the reactive intermediate scavenging agent reacts with reactive intermediates present in the environment, thereby inactivating the reactive intermediates and preventing them from reacting with other molecules. In certain embodiments, the reactive intermediate scavenging agent is oxidized as a result of reacting with reactive intermediates. In further embodiments, the reactive intermediate is reactive oxygen species (ROS) and/or reactive nitrogen species (RNS). In a specific embodiment, the reactive intermediate is a ROS and is hydrogen peroxide (H₂O₂).

As used herein, the term “reactive oxygen species” (ROS) refers to endogenously produced reactive small molecules containing at least one oxygen atom. Similarly, the term “reactive nitrogen species” (RNS) refers to endogenously produced reactive small molecules containing at least one nitrogen atom. In some embodiments, ROS include, but are not limited to, hydrogen peroxide (H₂O₂), superoxide (O₂.⁻) and hydroxyl radical (.OH). In some embodiments, RNS include, but are not limited to, nitric oxide (NO.), peroxynitrite (ONOO⁻), and nitrosoperoxycarbonate (ONOOCO₂⁻).

In some embodiments, the reactive intermediates are present in one or more bodily fluids of a subject afflicted with an oxo associated state, e.g., a hypermetabolic associated state. In some embodiments, the levels of reactive intermediates present in one or more bodily fluids of a subject afflicted with an oxo associated state, e.g., a hypermetabolic associated state are greater than the levels of reactive intermediates present in one or more bodily fluids of a healthy subject. Exemplary bodily fluids include, but are not limited to, urine, whole blood, blood serum, blood plasma, cerebrospinal fluid, saliva or lymph. In other embodiments, the reactive intermediates are present in exhaled breath of a subject afflicted with an oxo associated state, e.g., a hypermetabolic associated state. In some embodiments, the levels of reactive intermediates present in exhaled breath of a subject afflicted with an oxo associated state, e.g., a hypermetabolic associated state are greater than the levels of reactive intermediates present in exhaled breath of a healthy subject.

In some embodiments, the oxoprotective agent is a glutathione level restoring agent. Glutathione (GSH, L-gamma-glutamyl-L-cysteinylglycine) is a tripeptide co-factor that is present in animal cells and that has the following structure:

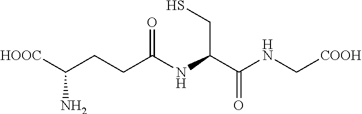 [SMILES: N[C@@H](CCC(=O)N[C@@H](CS)C(=O)NCC(=O)O)C(=O)O]

Glutathione is present at a concentration of about 5 mM in animal cells and acts as a reducing agent due to the presence of a thiol group. Glutathione serves as an electron donor and reduces disulfide bonds within cytoplasmic proteins to cysteines. In the process, glutathione is converted to its oxidized form glutathione disulfide (GSSG), also called L(-)-Glutathione.

Once oxidized, glutathione can be reduced back by glutathione reductase, using NADPH as an electron donor. The ratio of reduced glutathione to oxidized glutathione within a cell is a measure of the extent of oxidation being sustained by the cell. Accordingly, in some embodiments, the glutathione level restoring agent acts to increase the amount of reduced glutathione relative to the amount of oxidized glutathione. In some embodiments, the glutathione level restoring agent increases the amount of reduced glutathione relative to the amount of oxidized glutathione by neutralizing reactive oxygen species (ROS) and reactive nitrogen species (RNS).

In some embodiments, the oxoprotective agent reacts with ROS. In some embodiments, the reactive intermediate scavenging agent is oxidized as a result of reacting with reactive oxygen species (ROS). In certain embodiments, the glutathione level restoring agent increases the amount of reduced glutathione relative to the amount of oxidized glutathione by neutralizing reactive oxygen species (ROS). In a specific embodiment, the reactive oxygen species is H₂O₂.

In certain preferred embodiments, the oxoprotective agent is capable of being administered intravenously to a subject. In some embodiments, the intravenously administered oxoprotective agent is capable of neutralizing ROS and RNS, e.g., H₂O₂, in the subject's blood. Intravenous administration of the oxoprotective agent is preferred, because it delivers the oxoprotective agent to the blood of a subject more quickly and efficiently than e.g., oral administration. Moreover, a subject afflicted with an oxo associated state, e.g., a hypermetabolic associated state, may be too ill or even unconscious to take any medication, such as an oxoprotective agent, orally.

An oxo associated state is a state characterized by an increased level of reactive intermediate, e.g., ROS, and/or a diminished level of glutathione in a subject afflicted with an oxo associated state. In one embodiment, an oxo associated state is also a hypermetabolic associated state.

Hypermetabolism, or a hypermetabolic state, is a state characterized by increased metabolic activity. In some embodiments, a hypermetabolism can accompany pathological conditions that are characterized by upregulation of the immune system. Without wishing to be bound by a specific theory, it is believed that hypermetabolism provides the energy to sustain the highly upregulated immune function that may be switched on by the presence of a pathogen, such as a bacterium or a virus, or by non-infection related triggers. The abrupt global increase of cellular bioenergetics reactions to several times their normal basal state presents the cell with a surge of toxic metabolic by-products that must be neutralized to avoid accumulation and cell death. Hydrogen peroxide (H₂O₂), a reactive oxygen species, is a significant metabolic by-product that is generated in increased amounts when cellular processes such as protein synthesis, DNA recycling and ATP production are upregulated during periods of hypermetabolism that accompany systemic inflammation.

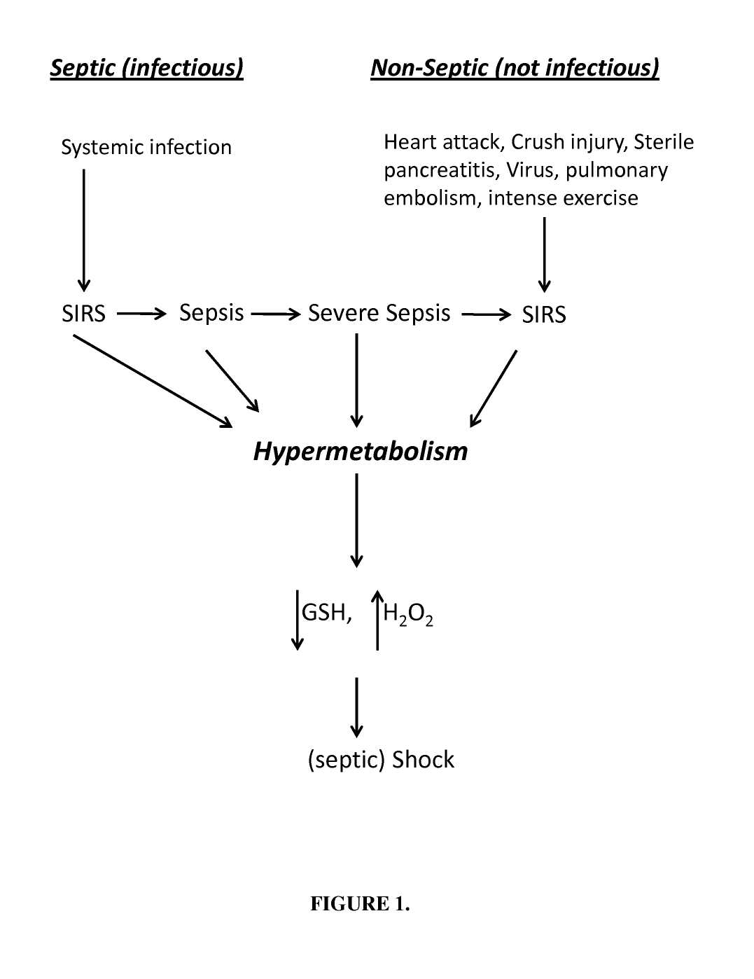

Without wishing to be bound by a specific theory, it is believed that pathogenesis of an oxo associated state, e.g., a hypermetabolic associated state, is initiated by depletion of glutathione as the crucial early event responsible for triggering a pathologic process in which H₂O₂ accumulates in tissues and blood of a subject, leading to microvascular dysfunction and organ failure. The proposed mechanism of pathogenesis of hypermetabolic associated states is illustrated in FIG. 1.

The majority of cellular H₂O₂ is neutralized by glutathione peroxidase (GPx), a selenium containing enzyme, which utilizes glutathione as a donor of reducing equivalents during the enzymatic conversion of H₂O₂ to water. Glutathione is consumed in this reaction and must be replenished in order to prevent accumulation of H₂O₂ within the cell. However, during periods of high H₂O₂ production, the availability of glutathione may be insufficient to keep up with the demand leading to net H₂O₂ accumulation and glutathione depletion resulting in severe cellular dysfunction that can affect any organ.

Excess H₂O₂ can easily diffuse out of parenchymal cells through capillary endothelium and into the blood. This augments endothelial generated H₂O₂ and results in oxidative damage and microangiopathic dysfunction. The inability to buffer cellular H₂O₂ signals a systemic failure of reductive (anti-oxidant) capacity as the excess oxidant load is discharged into the plasma, resulting in increased levels of H₂O₂ in the blood, or systemic oxidative stress. Over time, plasma reductive capacity is exhausted leading to severe disruption in plasma redox potential, which, as studies have shown, is strongly associated with an unfavorable outcome.

In the context of systemic oxidative stress, the anapleurosis of glutamine is a late effect of uncompensated cellular oxidative damage that contributes to the propagation of cellular oxidative stress. In high oxidative stress states (i.e., sepsis) beta oxidation is impaired due to the inhibition of intramitochondrial thiolase. Deprived of this energy source, the cell switches to the metabolism of glutamine via the Krebs cycle (anapleurosis), making the glutamate derived from glutamine unavailable for biosynthesis of glutathione that can alleviate oxidative stress. This contributes to a cycle of worsening oxidative stress that can lead to cell death. Without an external source of reducing equivalents, the cell cannot survive.

In some embodiments, the oxoprotective agent is an active sulfur compound. In certain embodiments, the term “active sulfur compound” excludes sodium 2-mercaptoethene sulfonate (mesna) or disodium 2,2′-dithiobis ethane sulfonate (dimesna). As used herein, the term “active sulfur compound” encompasses the following species: 1) sulfide compounds, 2) sulfite compounds, 3) thiosulfate compounds, 4) thionate compounds, 5) thionite compounds, 6) organic, inorganic or organometallic precursors of sulfide compounds, sulfite compounds, thiosulfate compounds, thionate compounds, and thionite compounds, 7) organic, inorganic or organometallic precursor compounds and 8) organic sulfur compounds.

Sulfide compounds are compounds formally containing the divalent Sₙ moiety (S=sulfur; n=1, 2, 3, etc.) chemically bonded to hydrogen and/or a metal (or metals) and/or a polyatomic cation (or cations). Examples of sulfide compounds include, but are not limited to, hydrogen sulfide, hydrogen disulfide, hydrogen tetrasulfide, sodium hydrosulfide, sodium hydrosulfide dihydrate, sodium sulfide, sodium sulfide nonahydrate, potassium sulfide, calcium sulfide, iron(II) sulfide, silicon (IV) sulfide, zinc sulfide, bismuth (III) sulfide, sodium disulfide, magnesium disulfide, iron (II) disulfide, sodium tetrasulfide, barium tetrasulfide, potassium pentasulfide, cesium hexasulfide, potassium iron (III) sulfide, ammonium sulfide, ammonium disulfide, ammonium tetrasulfide, and the like.

Sulfite compounds are compounds formally containing the divalent sulfite moiety (SO₃) chemically bonded to hydrogen, and/or a metal (or metals) and/or a polyatomic cation (or cations). Examples of sulfite compounds include, but are not limited to, sodium sulfite, potassium sulfite, ammonium sulfite, calcium sulfite, cesium hydrogen sulfite, and the like.

Thiosulfate compounds are compounds formally containing the divalent thiosulfate moiety (S₂O₃) chemically bonded to hydrogen and/or a metal (or metals) and/or a polyatomic cation (or cations). Examples of thiosulfate compounds include, but are not limited to, sodium thiosulfate (Na₂S₂O₃), potassium thiosulfate (K₂S₂O₃), sodium thiosulfate pentahydrate (Na₂S₂O₃*5H₂O), magnesium thiosulfate (MgS₂O₃), silver thiosulfate (Ag₂S₂O₃) and ammonium thiosulfate [(NH₄)₂S₂O₃].

Thionate compounds are compounds formally containing the divalent SₙO₆ (n>1) moiety chemically bonded to hydrogen and/or a metal (or metals) and/or a polyatomic cation (or cations). Examples of thionate compounds include, but are not limited to, calcium dithionate (CaS₂O₆), barium dithionate dihydrate (BaS₂O₆*2H₂O), sodium trithionate, sodium tetrathionate and the like.

Thionite compounds are compounds formally containing the divalent SnO₂ₙ (n=1 or 2) moiety chemically bonded to a hydrogen and/or a metal (or metals) and/or a polyatomic cation (or cations). Examples of thionite compounds include, but are not limited to, zinc sulfoxylate, zinc dithionite, sodium dithionite, sodium dithionite dihydrate, and the like.

Organic, inorganic or organometallic precursor compounds, as defined above, are any and all chemical species from which sulfide compounds and/or sulfite compounds and/or thiosulfate compounds and/or thionite compounds and/or thionate compounds, can arise through chemical change and/or enzyme action and/or biotransformation in a mammal's body. Therefore, tetraphosphorus decasulfide (P₄S₁₀), sodium thiosilicate (Na₂SiS₃) and elemental sulfur are precursors of sulfide compounds; whereas sodium metabisulfite (Na₂S₂O₅), diethyl sulfite and sodium sulfate are precursors of sulfite compounds.

Examples of organic sulfur compounds include, but are not limited to, sodium formaldehyde sulphoxylate, thio-urea, thiosorbitol, cysteine hydrochloride, cystine, cysteine, acetlycysteine, glutathione, cysteamine, methionine, thioglycerol, thioglycolic acid and thiolactic acid.

In a specific embodiment, the oxoprotective agent is a thiosulfate compound. In a further embodiment, the thiosulfate compound is sodium thiosulfate. Accordingly, in a preferred embodiment, the present invention provides methods for treating an oxo associated state, e.g., a hypermetabolic associated state, in a subject, comprising administering to said subject an effective amount of sodium thiosulfate. In a further embodiment, the sodium thiosulfate reacts with endogenously produced H₂O₂ present in the subject, thereby preventing H₂O₂ from reacting with other molecules and from causing cellular disruption and organ damage in the subject afflicted with the oxo associated state, e.g., a hypermetabolic associated state.

In other embodiments, the oxoprotective agent is selected from the group consisting of N-acetylcysteine, 3-tert-butyl-4-hydroxyanisole, 2,6-di-tert-butyl-p-cresol, tert-butylhydroquinone, caffeic acid, chlorogenic acid, cysteine, cysteine hydrochloride, decylmercaptomethyl-imidazole, diamylhydroquinone, di-tert-butylhydroquinone, dicetyl thiodipropionate, digalloyl trioleate, dilauryl thiodipropionate, dimyristyl thiodipropionate, dioleyl tocopheryl methylsilanol, disodium rutinyl disulphate, distearyl thiodipropionate, ditridecyl thiodipropionate, dodecyl gallate, edarovone, erythorbic acid, ethyl ferulate, ferulic acid, hydroquinone, p-hydroxyanisole, hydroxylamine hydrochloride, hydroxylamine sulphate, isooctyl thioglycolate, kojic acid, madecassicoside, methoxy-PEG-7-rutinyl succinate, mesalazine, methylene blue, nordihydrogualaretic acid, octyl gallate, phenylthioglycolic acid, phloroglucinol, propyl gallate, rosmarinic acid, rutin, sodium erythorbate, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocophersolan, tocopherol (e.g., vitamin E) and its derivatives (e.g., vitamin E derivatives such as vitamin E acetate, vitamin E linoleate, vitamin E nicotinate and vitamin E succinate), o-tolylbiguanide, tris(nonylphenyl)phosphite, dexpanthenol, alpha-hydroxycarboxylic acids (e.g., glycolic acid, lactic acid, mandelic acid) and salts thereof, p-hydroxybenzoic esters (e.g., methyl, ethyl, propyl or butyl esters thereof), dimethyloldimethylhydantoin, N-acylamino acids and salts thereof (e.g., N-octanoylglycine, Lipacide C8G) and hinokitol, metal borohydrides, sodium hydrosulfite, dimethylthiourea, sodium bisulfate, thiourea dioxide, diethylhydroxylamine, zinc dust, sodium cyanoborohydride, sodium hydride, trimethyl borate, benzyl triphenphosphonium chloride, butyl triphenphosphonium bromide, ethyl triphenphosphonium acid acetate, ethyl triphenphosphonium bromide, ethyl triphenphosphonium iodide, ethyl triphenphosphonium phosphate, tetrabutyl phosphonium acid acetate, as well as glutathione, or a monoester or diester glutathione, diester glutathione or multiester glutathione derivative.

In other embodiments, the oxoprotective agent may be an enzyme, such as catalase or glutathione peroxidate (GPx).

### Oxo Associated States

The present invention provides methods for diagnosing, treating or monitoring clinical progression of an oxo associated state, e.g., a hypermetabolic associated state, in a subject. In some embodiments, the method for treating an oxo associated state, e.g., a hypermetabolic associated state, in a subject comprises administering to said subject an effective amount of an oxoprotective agent, such that the oxo associated state in said subject is treated. In some embodiments, the subject is a human. In other embodiments, the subject is any non-human animal, such as a domestic pet, e.g., a cat, a dog, a horse, or a zoo animal. Accordingly, methods of the present invention are appropriate for veterinary use.

The term “oxo associated state” encompasses any condition associated with increased levels of reactive intermediates, such as ROS or RNA, and/or diminished levels of glutathione in a subject afflicted with an oxo associated state. In some embodiments, the oxo associated state is associated with an increased level of H₂O₂ and/or diminished level of glutathione. In some embodiments, an oxo associated state is a hypermetabolic associated state.

The term “hypermetabolic associated state” refers to any condition associated with hypermetabolism, or increased rate of metabolic activity. In some embodiments, the hypermetabolic associated state is characterized by a systemic hypermetabolic response. In some embodiments, the hypermetabolism is associated with upregulation of the immune system that can occur during an acute or a chronic immune response. In some embodiment, the hypermetabolic associated state is characterized by increased oxygen consumption; hyperthermia; increased nitrogen excretion; augmented catabolism of carbohydrates, proteins, and triglycerides in order to meet the increased metabolic demands. In some embodiments, the hypermetabolic associated state is characterized by higher than normal levels of hydrogen peroxide in the blood of a subject afflicted with a hypermetabolic associated state. In some embodiments, the hypermetabolic associated state is characterized by lower than normal levels of glutathione in the blood of a subject afflicted with a hypermetabolic associated state.

Examples of hypermetabolic associated states may include bacterial, viral or parasitic infections, sepsis and sepsis associated states, burns, trauma, fever, long-bone fractures, hyperthyroidism, prolonged steroid therapy, surgery, bone marrow transplants, pulmonary emboli, microangiopathic dysfunction, myocardial infarction, crush injuries, a viral infection or intense exercise.

In some embodiments, a hypermetabolic associated state may result from a bacterial infection. In some embodiments, the bacterial infection may follow trauma, crush or burn injuries. In certain embodiments, a hypermetabolic associated state that results from a bacterial infection is a sepsis associated state. In a specific embodiment, infectious endocarditis is an example of a hypermetabolic associated state resulting from a bacterial infection.

The term “sepsis associated state”, as used herein, refers to any one condition in the sepsis continuum, as defined by the American College of Chest Physicians and the Society of Critical Care Medicine (Bone et al. (1992) Chest, 101(6):1644-55, the entire contents of which are hereby incorporated herein by reference). In some embodiments, the term “sepsis associated state” can refer to any one of Systemic Inflammatory Response Syndrome (SIRS), Sepsis, Severe Sepsis and Septic Shock.

Systemic Inflammatory Response Syndrome (SIRS) is defined by the presence of two or more of the following: a) hypothermia or fever; b) increased heart rate (>90 beats per minute); c) tachypnea or hypocapnia due to hyperventilation; and d) leukopenia, leukocytosis, or bandemia.

Sepsis is defined as SIRS in response to a confirmed infectious process. Infection can be suspected or proven (e.g., by culture, stain, or polymerase chain reaction), or a clinical syndrome pathognomonic for infection. Specific evidence for infection includes white blood cells (WBCs) in normally sterile fluid (such as urine or cerebrospinal fluid); evidence of a perforated viscus (free air on abdominal x-ray or CT scan; signs of acute peritonitis); abnormal chest x-ray (CXR) consistent with pneumonia (with focal opacification); or petechiae, purpura, or purpura fulminans.

Severe Sepsis is defined as sepsis with organ dysfunction, hypoperfusion, or hypotension.

Septic Shock is defined as sepsis with refractory arterial hypotension or hypoperfusion abnormalities in spite of adequate fluid resuscitation. Signs of systemic hypoperfusion may be either end-organ dysfunction or increased serum lactate (>4 mmol/L). Other signs include oliguria and altered mental status. Patients are defined as having septic shock if they have sepsis plus hypotension after aggressive fluid resuscitation (typically upwards of 6 liters or 40 ml/kg of crystalloid solution).

In some embodiments, the definitions of sepsis associated states, e.g., Systemic Inflammatory Response Syndrome (SIRS), Sepsis, Severe Sepsis and Septic Shock, as defined by the American College of Chest Physicians and the Society of Critical Care Medicine also encompass modifications for pediatric population, e.g., as described in Goldstein et al. (2005) Pediatr. Crit. Care Med., 6(1):2-8, the entire contents of which are hereby incorporated herein by reference.

In some embodiments, the hypermetabolic associated state may result from a viral infection. In further embodiments, the hypermetabolic associated state is Dengue fever, Dengue hemorrhagic fever or a Dengue shock syndrome. In a specific embodiment, the viral infection is Dengue fever.

In some embodiments, the hypermetabolic associated state does not result from a bacterial or a viral infection. Examples of hypermetabolic states that are not associated with an infection may include trauma, crush or burn injuries, myocardial infarction, or an inflammatory state, such as pancreatitis.

In some embodiments, the systemic hypermetabolic response may lead to a decrease in or a depletion of glutathione. In some embodiments, systemic hypermetabolic response may lead to accumulation of one or more reactive intermediates, such as H₂O₂.

In some embodiments, the present invention also provides methods for screening, diagnosing, monitoring the clinical progression of, or assessing therapy for an oxo associated state, e.g., a hypermetabolic associated state or a sepsis associated state, in a subject, the method comprising measuring the level of one or more reactive intermediates in one or more bodily fluids or exhaled breath of said subject. In other embodiments, the present invention also provides methods of determining a subject's risk for developing an oxo associated state, e.g., a hypermetabolic associated state or a sepsis associated state, the method comprising measuring the level of one or more reactive intermediates in one or more bodily fluids or exhaled breath of the subject. In some embodiments, the subject is a human. In other embodiments, the subject is any non-human animal, such as a domestic pet, e.g., a cat, a dog, a horse, or a zoo animal. In some embodiments, the bodily fluid is urine, whole blood, blood serum, blood plasma, cerebrospinal fluid, saliva or lymph.

In some embodiments, the present invention also provides methods for screening, diagnosing, monitoring the progression of, or assessing therapy for an oxo associated state, e.g., a hypermetabolic associated state or a sepsis associated state, in a subject, the method comprising measuring the level of glutathione in one or more bodily fluids or in exhaled breath of said subject. In other embodiments, the present invention also provides methods of determining a subject's risk for developing an oxo associated state, e.g., a hypermetabolic state or a sepsis associated state, the method comprising measuring the level of glutathione in one or more bodily fluids or in exhaled breath of a subject. In some embodiments, the subject is a human. In other embodiments, the subject is a non-human animal, such as a domestic pet, e.g., a cat, a dog, a horse, or a zoo animal. Accordingly, methods of the present invention are appropriate for veterinary use. In some embodiments, the bodily fluid is urine, whole blood, blood serum, blood plasma, cerebrospinal fluid, saliva or lymph.

In further embodiments, the method comprises comparing the level of one or more reactive intermediates in a bodily fluid or exhaled breath of a subject with the level of the same one or more reactive intermediates present in a bodily fluid or exhaled breath of a subject who is not afflicted with an oxo associated state, e.g., a hypermetabolic associated state. In some embodiments, the reactive intermediate is H₂O₂, the bodily fluid is whole blood, blood serum or blood plasma, and the measuring is accomplished with a pin prick of blood onto the appropriate chemical reagent or by using methods known in the art. In some embodiments, the reactive intermediate is H₂O₂, the bodily fluid is urine, and the measuring is accomplished via a urinary dipstick or by using methods known in the art. In some embodiments, the reactive intermediate is H₂O₂, the bodily fluid is saliva, and the measuring is accomplished via an oral swab or by using methods known in the art.

In some embodiments, the method comprises comparing the level of glutathione in a bodily fluid or exhaled breath of a subject with the level of the same one or more reactive intermediates present in a bodily fluid or exhaled breath of a subject who is not afflicted with a hypermetabolic associated state. In some embodiments, the bodily fluid is whole blood, blood serum or blood plasma, and the measuring is accomplished by using methods known in the art.

“Treating an oxo associated state, e.g., a hypermetabolic associated state or a sepsis associated state, in a subject” includes achieving, partially or substantially, one or more of the following: ameliorating or improving a clinical symptom or indicator associated with a hypermetabolic associated state (such as tissue or serum components); arresting the progression or worsening of the oxo associated state, e.g., a hypermetabolic associated state. In some embodiments, treating an oxo associated state, e.g., a hypermetabolic associated state, or, more specifically, a sepsis associated state, includes arresting the progression from SIRS to Sepsis, from Sepsis to Severe Sepsis or from Severe Sepsis to Septic Shock. In other embodiments, treating an oxo associated state, e.g., a hypermetabolic associated state, includes increasing short-term or long-term survival of a subject afflicted with the oxo associated state, e.g., a hypermetabolic associated state.

In certain embodiments, treating an oxo associated state, e.g., a hypermetabolic associated state or a sepsis associated state, comprises decreasing the level of one or more reactive intermediates, such as H₂O₂, in a subject over time. In a specific embodiment, the treatment comprises decreasing the levels of H₂O₂ in the subject's blood over time.

In other embodiments, treating an oxo associated state, e.g., a hypermetabolic associated state or a sepsis associated state, comprises increasing the level of glutathione in the subject's blood over time. In some embodiments, treating an oxo associated state, e.g., a hypermetabolic associated state, or, more specifically, a sepsis associated state, in a subject comprises maintaining in the subject blood pressure levels that are compatible with life. In a further embodiment, treating an oxo associated state, e.g., a hypermetabolic associated state or a sepsis associated state, in a subject is associated with achieving in the subject blood pressure levels that are close to the blood pressure levels in a healthy individual.

The term “effective amount” is the quantity of an oxoprotective agent required to maintain a desired concentration of an oxoprotective agent in a subject while being effective for treating an oxo associated state, e.g., a hypermetabolic associated state. In some embodiments, the effective amount of oxoprotective agent is sufficient to maintain the desired plasma or serum concentration of the oxoprotective agent. The precise amount of the oxoprotective agent to be administered to a subject will depend on the oxoprotective agent levels that are to be achieved and/or maintained in a subject, as well as on the exact mode of administration. The levels of the oxoprotective agent to be achieved and/or maintained in the subject may be assessed by periodic monitoring using laboratory tests that are known in the art. When oxoprotective agent is co-administered with, e.g., another oxoprotective agent or with alpha-lipoic acid, as is also discussed below, for the treatment of an oxo associated state, e.g., a hypermetabolic associated state, an “effective amount” of the oxoprotective agent may need to be adjusted and/or maintained. Suitable dosages for oxoprotective agents of the invention are known and can be adjusted by the skilled artisan according to the condition of the subject and the oxoprotective agent being used.

The terms “administer”, “administering” or “administration” include any method of delivery of a pharmaceutical composition or one or more agents into a subject's system or to a particular region in or on a subject. In certain embodiments of the invention, the one or more agents can be administered intravenously, intramuscularly, subcutaneously, intradermally, intranasally, orally, transcutaneously, mucosally, or via inhalation. In some embodiments, the one or more agents can be administered via an endotracheal tube if the subject is on a ventilator. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, etc.

In some embodiments, treatment of an oxo associated state, e.g., a hypermetabolic associated state, is administered by extracorporeal circulation, e.g., by using an extracorporeal device comprising a filter impregnated with one or more oxoprotective agents that can be used in the methods of the present invention. In some embodiments, the oxoprotective agent in the filter neutralizes and/or removes hydrogen peroxide present in the blood of a subject afflicted with the oxo associated state, e.g., a hypermetabolic associated state.

In some embodiments, administration of an oxoprotective agent is parenteral. In a preferred embodiment, the administration is intravenous. In an even more preferred embodiment, the oxoprotective agent is administered by slow intravenous infusion. In a further embodiment, the oxoprotective agent administered by slow intravenous infusion is sodium thiosulfate.

For parenteral administration, the oxoprotective agent may be dissolved in a suitable solvent for intravenous administration to produce a solution which may be injected or infused. One or more pharmaceutically acceptable excipients may also be added.

In some embodiments, the oxoprotective agent, such as sodium thiosulfate, is administered as a monotherapy, e.g., as the only oxoprotective agent administered to a subject for treating an oxo associated state, e.g., a hypermetabolic associated state. In other embodiments, the oxoprotective agent may be administered in combination with one or more second agents. In further embodiments, the oxoprotective agent is administered in combination with a second oxoprotective agent. In some embodiments, the second oxoprotective agent is an active sulfur compound. In a specific embodiment, the second oxoprotective agent is N-acetylcysteine. Accordingly, in a preferred embodiment, sodium thiosulfate is administered in combination with N-acetylcysteine for treating an oxo associated state, e.g., a hypermetabolic associated state, in a subject.

As used herein, the term “administered in combination” implies that two or more agents may be administered at the same time or less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. Additional agents may be administered intravenously, intramuscularly, subcutaneously, intradermally, intranasally, orally, transcutaneously, mucosally or via inhalation, by the same route or different route as the first agent. Moreover, two or more agents may be administered as a part of the same pharmaceutical composition or as parts of different pharmaceutical compositions. Appropriate administration regimens for two or more agents to be administered in combination will be apparent to one of skill in the art.

In another preferred embodiment, the first oxoprotective agent, such as sodium thiosulfate, and the second oxoprotective agent, such as N-acetylcysteine, are both administered as parts of the same pharmaceutical composition. In a further embodiment, the administration is intravenous administration, e.g., a slow intravenous infusion.

In a specific embodiment, sodium thiosulfate is administered to adults and adolescents at an intravenous dose of 12.5 grams, administered at a rate of, for instance, 0.625 to 1.25 grams (2.5 to 5 mL) per minute. In other embodiments, sodium thiosulfate may be administered to children at a dose of 250 mg/kg or approximately 7 g/m² of body surface area via slow intravenous injection or an intravenous infusion at a rate of 2.5 to 5 mL/minute.

In another specific embodiment, N-acetylcysteine is administered to adults at the loading dose of 150 mg/kg in 200 mL of a formulation also comprising 5% dextrose and administered intravenously over 15 to 30 minutes. The loading dose is followed by a first maintenance dose of 50 mg/kg in 500 mL of a formulation comprising 5% dextrose administered intravenously over 4 hours. The first maintenance dose is followed by a second maintenance dose of 100 mg/kg in 1000 mL of a formulation comprising 5% dextrose that is administered intravenously over prolonged period, for instance, 16 hours.

Adjustments of N-acetylcysteine dose may be required for children and patients at risk for fluid overload. In patients weighing less than 30 kg, 20% N-acetylcysteine may be diluted to a final concentration of 40 mg/mL. This may be accomplished by adding 50 mL (10 g) of 20% solution of N-acetylcysteine (Acetadote®) to 200 mL of D5W by removing 50 mL from a 250 mL bag. This single bag can be used for the entire infusion. The loading dose may be infused intravenously at the dose of 3.75 mL/kg (or 150 mg/kg), for instance, over 15 to 30 minutes. The loading dose may be followed by a first maintenance dose of 1.25 mL/kg (or 50 mg/kg) over 4 hours (or 0.31 mL/kg/hr), and a second maintenance dose of 2.5 mL/kg (or 100 mg/kg), for instance, over the next 16 hours (0.16 mL/kg/hr).

In some embodiments, one or more oxoprotective agents are administered in combination with alpha-lipoic acid (ALA). ALA is a small molecule (MW 206.3, CAS #1077-28-7). It is known by a variety of names which vary depending upon its redox state (oxidized or reduced) and the enantiomeric configuration around the number three carbon chiral center (*). The older relative (comparison) based D and L nomenclature has been replaced by the designation R and S indicating absolute stereochemical configuration.

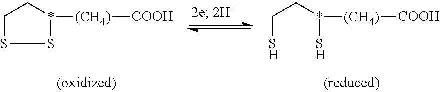

ALA is an eight carbon cyclic disulfide containing fatty acid which is synthesized in trace amounts within mitochondria in all cells of the body. In its natural state ALA is covalently bonded, via its terminal carboxyl in an amide linkage, to the epsilon amino group of lysine residues which form part of multi-subunit enzyme complexes that catalyze vital energy metabolism reactions within mitochondria. There is very little free ALA in the cytoplasm or circulation.

The bonding of ALA to its cognate protein is accomplished as a post-translational modification of the enzyme. In its protein bound state it is a required enzymatic co-factor called lipoamide. The enzyme complexes which use ALA are the pyruvate dehydrogenase complex which catalyzes the conversion of pyruvate to acetyl-CoA, a vital substrate for energy production via the Krebs (citric acid) cycle. The alpha-ketoglutarate complex which catalyzes another important Krebs cycle reaction, the branched chain alpha-keto acid dehydrogenase complex which catalyzes the oxidative decarboxylation of three branched chain amino acids (valine, leucine and isoleucine) generating acetyl-CoA for entry into the Krebs cycle and finally the glycine cleavage system complex that catalyzes the formation of 5,10 methylene tetrahydrofolate which plays a vital role in the synthesis of nucleic acids.

The natural function of ALA is to bind and transfer acyl groups to successive enzymatic active sites among the subunits of each enzyme complex. In this process of acyl transfer ALA is reduced to dihydrolipoic acid and subsequently re-oxidized back to ALA by its attached cognate enzyme which readies it for the next acyl transfer. ALA has a high degree of bioavailability after oral administration and exhibits both lipid and water solubility. This allows its distribution to both intra and extra cellular compartments.

In some embodiments, the ALA is R-dihydro-lipoic acid. ALA may be administered intravenously, intramuscularly, subcutaneously, intradermally, intranasally, orally, transcutaneously, mucosally or via inhalation. In one embodiment, ALA is administered orally. In another embodiment, ALA is administered intravenously. In another embodiment, ALA is inhaled.

In one embodiment, an oxoprotective agent, e.g., sodium thiosulfate, is administered in combination with R-dihydro-lipoic acid, wherein administration of sodium thiosulfate is intravenous and administration of R-dihydro-lipoic acid is oral. In another embodiment, an oxoprotective agent, e.g., sodium thiosulfate, is administered in combination with the second oxoprotective agent, e.g., N-acetylcysteine and a third oxoprotective agent, e.g., R-dihydro-lipoic acid. In a further embodiment, sodium thiosulfate and N-acetylcysteine are administered intravenously as a part of the same pharmaceutical composition and R-dihydro-lipoic acid is administered orally.

For oral administration, ALA may be combined with one or more pharmaceutically acceptable excipients, fillers and/or diluents. Oral dosage forms may include pills, caplets, tablets, and the like. Alternatively, ALA may be contained in a swallowable container such as a gelatin capsule or the like. ALA may be administered to adults in oral dosages of 600-900 mg daily in 2-3 divided doses. ALA may be administered to children in oral dosages of 5 mg/kg in 2-3 daily divided doses. Studies have shown that ALA is safe at doses up to 2 grams daily for 2 years in humans. In some embodiments, ALA may be administered in a dose of 300 mg twice daily, and the dose may be increased up to 900 mg.

In at least some embodiments, the methods of the invention for treating an oxo associated state, e.g., a hypermetabolic associated state, is used alone, i.e., in the absence of other therapies for treating an oxo associated state. In other embodiments, the methods of the invention may be combined with other methods known and used in the art to treat a specific oxo associated state, e.g., a hypermetabolic associated state. For example, the methods of the invention may be combined with antibiotic therapy for treating sepsis associated states. In other embodiments, methods of the present invention may be combined with anti-viral therapies for treating a hypermetabolic associated state that may result from a viral infection. In a specific embodiment, the method of the present invention is administered in combination with a therapy with an anti-viral antibody for treating Dengue fever, Dengue hemorrhagic fever or Dengue shock syndrome.

### INCORPORATION BY REFERENCE

The contents of all references, patents, pending patent applications and published patents, cited throughout this application are hereby expressly incorporated by reference.

### EXEMPLIFICATION OF THE INVENTION

The invention will be further understood by the following examples. However, those skilled in the art will readily appreciate that the specific experimental details are only illustrative and are not meant to limit the invention as described herein, which is defined by the claims which follow thereafter.

### Example 1

### Peroxide Levels in Serum of Sepsis Patients

Samples from a total of 15 patients were analyzed. Of these, 14 patients were diagnosed with septic shock and one patient did not have septic shock and represented negative control. Blood samples were taken from the 14 patients at the time of sepsis diagnosis and at follow-up on day 2, 7, 14, 21 and 28 during hospitalization, and blood was immediately centrifuged and stored at −70° C. until future use. Serum peroxide levels were measured in each sample at the time of sepsis diagnosis and on each of the above days using the PEROXsay™ assay (G-Biosciences) that measures the oxidation of ferrous (Fe²⁺) ions to ferric (Fe³⁺) ions.

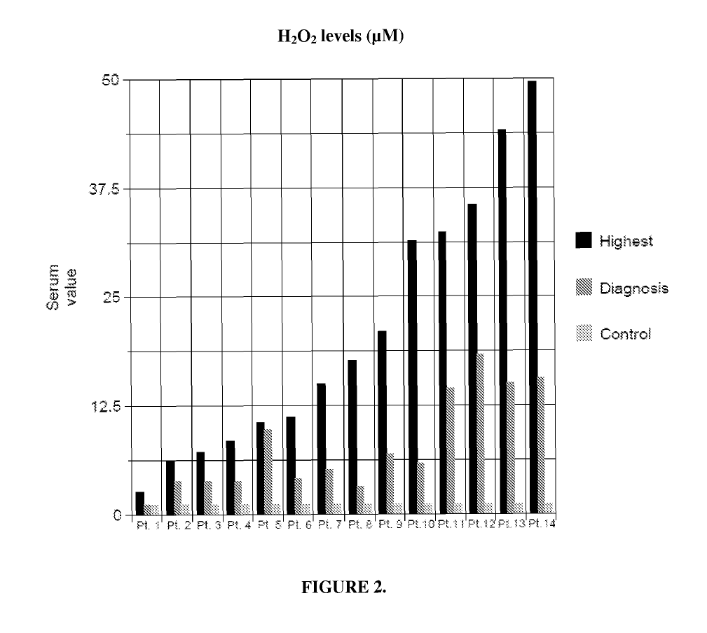

The results are presented in the table below and in FIG. 2.

Peroxide Concentration (μM)

| Patient | At | Highest Measured |
|---|---|---|
| No. | Diagnosis | Peroxide Concentration |
| 213 | 1.2 | 2.7 |
| 302 | 3.8 | 6.3 |
| 209 | 3.8 | 7.1 |
| 217 | 3.8 | 8.4 |
| 207 | 9.7 | 10.6 |
| 216 | 4.1 | 11.3 |
| 212 | 5.1 | 15.0 |
| 301 | 3.2 | 17.7 |
| 210 | 6.9 | 21.0 |
| 215 | 5.9 | 31.3 |
| 218 | 14.6 | 32.4 |
| 303 | 18.3 | 35.6 |
| 204 | 15.1 | 44.1 |
| 202 | 15.6 | 49.6 |
| Control | 1.15 | |

Compared to the normal control (1.15 μM), serum hydrogen peroxide was abnormally elevated in all sepsis patients at diagnosis (range 1.2-18.6 μM; mean 7.93 μM), and remained elevated throughout the study, during which the highest level was recorded (range 2-49.6 μM; mean 20.93 μM). This demonstrates that peroxide levels are elevated during sepsis and septic shock.

### Example 2

### Dengue Shock Syndrome

A subject presents with sudden high fever, severe headache and pain behind the eyes accompanied by severe muscle and joint aches, along with abdominal pain. Physical examination reveals that the subject is lethargic and poorly responsive to verbal commands, has a weak pulse, low blood pressure and labored breathing. Examination also reveals that the subject is cyanotic with blueness around the mouth, has blood spots in the skin (petechiae) and bleeding gums. The subject develops persistent vomiting, bleeding from the nose and gums and shortness of breath. The subject's blood pressure continues to decline and cannot be raised by intravenous fluids or vasopressor agents. A diagnosis of Dengue shock syndrome is made.

Peroxide and glutathione levels are measured in the subject. The measurement reveals higher than normal serum peroxide levels and lower than normal serum glutathione levels. Therapeutic doses of sodium thiosulfate, N-acetyl cysteine and R-dihydro lipoic acid (ALA) are administered as a treatment. As a result of the treatment, the subject's blood pressure gradually returns to normal, breathing is improved and the subject becomes alert and oriented. Cyanosis, abdominal pain and vomiting revolve as a result of the treatment, and the subject's condition returns to normal.

### Example 3

### Bacterial Septic Shock in a Diabetic

A subject presents with chills, fever and shortness of breath. The subject is diabetic and has had dental work prior to the onset of the symptoms. The subject appears lethargic. Physical examination reveals an increased heart rate, a high temperature and a heart murmur. The subject's blood pressure is low and unresponsive to intravenous fluids or vasopressor agents, and chest X-ray reveals an enlarged heart. Laboratory tests also reveal high white blood cell count, elevated liver enzymes and decreased renal function, moderate aortic valve regurgitation with vegetations, and a blood culture positive for bacteria.

A diagnosis of infective endocarditis with septic shock is made and the subject is started on antibiotics. However, the subject continues to deteriorate, becomes unresponsive and develops bleeding from gums and rectum, characteristic of diffuse intravascular coagulation (DIC).

Peroxide and glutathione levels are measured in the subject. The measurement reveals higher than normal serum peroxide levels and lower than normal serum glutathione levels. Therapeutic doses of sodium thiosulfate, N-acetyl cysteine and R-dihydro lipoic acid (ALA) are administered. Following treatment, the subject's blood pressure normalizes, temperature returns to normal and the bleeding stops. The subject is alert and responsive. Subject's repeat blood cultures are negative for bacteria.

### Example 4

### Crush Injury and Septic Shock

Subject presents after suffering a crush injury to the legs, with both lower extremities appearing contused and edematous. The subject's temperature is elevated, and the respiration is labored. The subject develops renal failure, high fever, high white blood cell count, and increased heart and respiratory rates. The subject's hypotension is unresponsive to intravenous fluids or vasopressor agents. The subject's blood cultures are positive for bacterial infection.

A diagnosis of septic shock subsequent to crush injury is made, and the subject is started on antibiotics. However, the subject continues to deteriorate and becomes lethargic and unresponsive. Measurement of peroxide and glutathione levels in the serum of the subject reveals higher than normal serum peroxide and lower than normal glutathione. Therapeutic doses of sodium thiosulfate, N-acetylcysteine and ALA are administered as treatment of septic shock due to crush injury. The subject's blood pressure increases towards normal, and the subject's temperature improves towards normal. Serum glutathione increases towards normal values and serum peroxide decrease toward normal values. The subject's blood pressure and temperature return to normal several days after the start of the treatment.

### Example 5

### Puerperal Septic Shock

A female subject presents with high fever, rapid heart rate and rapid shallow respiration after having given birth a day before. The subject is disoriented, and the subject's blood pressure is low. Laboratory tests reveal an elevated white blood cell count, elevated liver enzymes and decreased renal function, indicative of organ failure. A blood culture is positive for bacterial infection.

A diagnosis of puerperal (pregnancy related) septic shock is made and the subject is administered antibiotics. Despite the treatment, the subject develops cardiac arrest and is resuscitated. Laboratory results reveal a low serum glutathione level and a high serum peroxide level.

Therapeutic doses of sodium thiosulfate, N-acetylcysteine and ALA are administered as treatment of puerperal septic shock. The patient regains consciousness as a result of the treatment, and the blood pressure, temperature and white blood cell count normalize. The subject's serum glutathione increases towards normal and serum peroxide decreases towards normal after treatment.

### Example 6

### Sterile Pancreatitis with Shock

A subject presents in acute distress with severe nausea, vomiting and abdominal pain radiating to the subject's back. Assessment of vital signs reveals increased blood pressure and heart rate, rapid shallow respiration and high temperature. Laboratory tests reveal a high white blood cell count and elevated serum lipase and amylase. An abdominal CAT scan reveals pancreatic edema and enlargement. The subject is diagnosed with acute pancreatitis accompanied by a systemic inflammatory response syndrome (SIRS) and a hypermetabolic state.

The subject is administered intravenous fluids and vasopressor agents, but is unresponsive to the treatment. Additional lab results reveal early kidney and liver failure, while blood cultures are negative for bacterial infection.

Peroxide levels and glutathione levels in the serum of the subject are measured. The measurements reveal higher than normal serum peroxide levels and lower than normal serum glutathione levels. The subject is administered therapeutic doses of sodium thiosulfate, N-acetylcysteine and ALA. After the treatment, the subject's blood pressure increases and the subject experiences decreased abdominal pain. The subject's blood pressure ultimately returns to normal and the abdominal pain subsides. The subject's serum glutathione levels increase towards normal and serum peroxide levels decrease towards normal after treatment.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating an oxo associated state in a subject, the method comprising administering to said subject an effective amount of an oxoprotective agent, such that the hypermetabolic associated state in said subject is treated.

2. The method of claim 1, wherein the oxo associated state is a hypermetabolic associated state.

3. The method of claim 2, wherein the oxoprotective agent is a reactive intermediate scavenging agent.

4. The method of claim 2, wherein the oxoprotective agent is a glutathione level restoring agent.

5. The method of claim 4, wherein the oxoprotective agent is an active sulfur compound.

6. The method of claim 5, wherein the active sulfur compound is selected from a group consisting of a sulfide compound, a sulfite compound, a thiosulfate compound, a thionite compound, a thionate compound, an organic sulfur compound, or precursors, hydrates and mixtures thereof.

7. The method of claim 6, wherein the active sulfur compound is a thiosulfate compound.

8. The method of claim 7, wherein the thiosulfate compound is sodium thiosulfate, ammonium thiosulfate, calcium thiosulfate, potassium thiosulfate, silver thiosulfate, choline thiosulfate, gold sodium thiosulfate, magnesium thiosulfate hexahydrate, or thiosulfate hyposulfite.

9. The method of claim 8, wherein the active sulfur compound is sodium thiosulfate.

10. The method of claim 2, wherein the oxoprotective agent is administered in combination with alpha-lipoic acid.

11. The method of claim 10, wherein the alpha-lipoic acid is R-dihydro lipoic acid.

12. The method of claim 2, wherein the oxoprotective agent is administered parenterally.

13. The method of claim 12, wherein the oxoprotective agent is administered intravenously.

14. The method of any one of claims 10-13, wherein the alpha-lipoic acid is administered orally.

15. The method of any one of claims 10-13, wherein the oxoprotective agent and the alpha-lipoic acid are administered as a part of the same pharmaceutical composition.

16. A method for treating an oxo associated state in a subject, the method comprising administering to said subject an effective amount of a first oxoprotective agent in combination with a second oxoprotective agent, such that the hypermetabolic associated state in said subject is treated.

17. The method of claim 16, wherein the oxo associated state is a hypermetabolic state.

18. The method of claim 16, wherein the first oxoprotective agent and the second oxoprotective agent are both active sulfur compounds.

19. The method of claim 18, wherein the first oxoprotective agent and the second oxoprotective agent are each active sulfur compounds independently selected from the group consisting of a sulfide compound, a sulfite compound, a thiosulfate compound, a thionite compound, a thionate compound, an organic sulfur compound, or precursors, hydrates and mixtures thereof.

20. The method of claim 19, wherein the first oxoprotective agent is a thiosulfate compound and the second oxoprotective agent is an organic sulfur compound.

21. The method of claim 20, wherein the first oxoprotective agent is sodium thiosulfate and the second oxoprotective agent is N-acetylcysteine.

22. The method of any one of claims 16-21, wherein the first oxoprotective agent and the second oxoprotective agent are both administered parenterally.

23. The method of claim 22, wherein the first oxoprotective agent and the second oxoprotective agent are both administered intravenously.

24. The method of any one of claims 16-23, wherein the first oxoprotective agent and the second oxoprotective agent are administered as separate pharmaceutical compositions.

25. The method of any one of claims 16-23, wherein the first oxoprotective agent and the second oxoprotective agent are administered as parts of the same pharmaceutical composition.

26. The method of any one of claims 16-25, wherein the first and the second oxoprotective agents are administered in combination with alpha-lipoic acid.

27. The method of claim 26, wherein the alpha-lipoic acid is R-dihydro lipoic acid.

28. The method of claim 26, wherein the alpha-lipoic acid is administered orally.

29. The method of any one of claims 2-15 and 17-28, wherein the hypermetabolic associated state is a sepsis associated state.

30. The method of claim 29, wherein the sepsis associated state is Systemic Inflammatory Response Syndrome (SIRS).

31. The method of claim 29, wherein the sepsis associated state is sepsis.

32. The method of claim 29, wherein the sepsis associated state is severe sepsis.

33. The method of claim 29, wherein the sepsis associated state is septic shock.

34. A method for treating a hypermetabolic associated state in a subject, the method comprising administering to said subject an effective amount of sodium thiosulfate in combination with R-dihydro lipoic acid, such that the hypermetabolic associated state in said subject is treated.

35. A method for treating a hypermetabolic associated state in a subject, the method comprising administering to said subject an effective amount of sodium thiosulfate in combination with N-acetylcysteine and R-dihydro lipoic acid, such that the hypermetabolic associated state in said subject is treated.

36. A method for diagnosing an oxo associated state in a subject suspected of having the oxo associated state, the method comprising measuring the level of glutathione and/or the level of hydrogen peroxide in one or more bodily fluids or exhaled breath of the subject.

37. A method for monitoring treatment of an oxo associated state in a subject being treated for the oxo associated state, the method comprising measuring the level of glutathione and/or the level of hydrogen peroxide in the subject.

38. The method of claim 36 or claim 37, wherein the oxo associated state is a hypermetabolic associated state.

39. The method of claim 38, wherein measurement of the level of glutathione and/or the level of hydrogen peroxide is repeated at least once during administration of the treatment.

40. The method of claim 38, wherein the level of glutathione measured in a subject afflicted with a hypermetabolic associated state is lower than the level of glutathione in a healthy subject.

41. The method of claim 38, wherein the level of hydrogen peroxide measured in a subject afflicted with a hypermetabolic associated state is higher than the level of glutathione in a healthy subject.

42. A method for monitoring treatment of a hypermetabolic associated state in a subject, the method comprising measuring the level of one or more oxoprotective agents in the blood of said subject.

43. The method of claim 42, wherein the oxoprotective agent is sodium thiosulfate, N-acetylcysteine or R-dihydro lipoic acid.
